(19) United States
(12) Patent Application Publication
(10) Pub. No.: US 2011/0152647 A1
(43) Pub. Date: Jun. 23, 2011

(54) DIODE LASER DEVICE FOR THE NON-INVASIVE MEASUREMENT OF GLYCAEMIA

(75) Inventors: PIETRO TROMBETTA, Montano Lucino (CO) (IT); Vittorio Londoni, Bergamo (IT)

(73) Assignee: PIGNOLO S.P.A., Bergamo (IT)

(21) Appl. No.: 13/014,998

(22) Filed: Jan. 27, 2011

Related U.S. Application Data
(63) Continuation-in-part of application No. PCT/IT2008/000516, filed on Jul. 30, 2008.

(51) Int. Cl. A61B 5/1455

(52) U.S. Cl. 600/316

(57) ABSTRACT

A device has two diode laser sources in a range between 500 and 1000 nm, with a power in the range between 0.01 to 100 mW. Rays from the sources are conveyed through an optical condenser and, starting up a key, are emitted either on a nail or on the skin or even on a free blood sample. A photodiodic sensor or a CPU read the energy subtracted by glycate haemoglobin and free plasmic glucose. This value is converted into the immediate glycaemia value and appears on a device display.

This is a Continuation-in-Part application of International Application No. PCT/IT2008/000516, filed Jul. 30, 2008, which applicants herewith claim the benefit of priority under Title 35, U.S.C. §119 and §120. The content of PCT/IT2008/000516 is incorporated by reference herein.

The device is built using two laser diode sources, with a conventional or with a drugged fibre source, fed either through the electric system in case the device is in its fixed form or by (rechargeable) batteries for the portable, pocket-size device.

The two laser diode sources involved are in a range between 500 and 1000 nm, with a power in the range between 0.01 to 100 Mw.

The rays originating from the two laser diodes are conveyed through an optical condenser and, starting up a key, they are emitted either on a nail or on the skin or even on a free blood sample.

A photodiode sensor or a CPU read the value of the radiation energy that is subtracted by the glycate haemoglobin and free plasmic glucose present in the blood. This value is converted into the immediate glycaemia value and appears on a device display.

The device can store in its memory up to 5000 of these values.

The device is provided with a cable interface, IR and Bluetooth to connect and transmit the data to a PC.

The device allows to determine the glycaemia value without pricking the skin and without taking a blood sample.

### BRIEF DESCRIPTION OF THE DRAWINGS

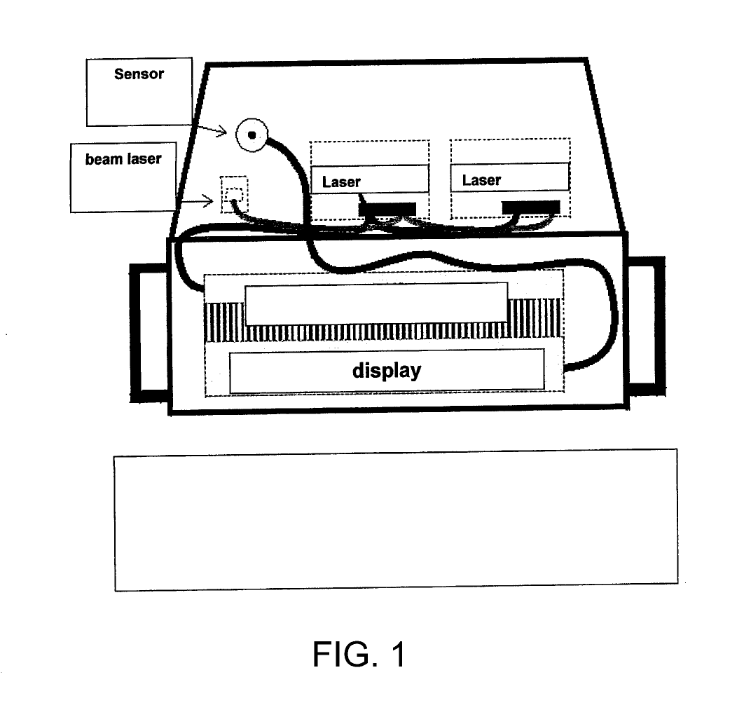

The only figure in the drawings, FIG. 1, is a schematic view of the invention.

### DESCRIPTION OF THE PREFERRED EMBODIMENT

More in detail, and with reference to FIG. 1, in the use and working of the present device, the patient simply has to put a finger into the device, operating a lever that opens a four-partitioned diaphragm.

Then, upon releasing the lever, a spring lets the diaphragm close, thus determining a centred arrangement of the finger, no matter its dimensions, on the focus of the two laser rays directed towards the photodiodes. (The laser device meets the requirements of Class 1 safety features).

The device uses two laser diode sources between 500 and 1000 nm.

The patient switches the device on and the passing of the finger sets off an optical barrier that controls the laser starting.

Then the photodiodes read the values of the laser radiation resulting from the selective absorption of the two laser rays, radiation energy by the glycate haemoglobin and free plasmic glucose present in the blood of the finger.

Moreover an analogic stage forwards these values to the processor that, after the stabilization of the system, digitises and normalises them, extracts a high number of them, strikes an average, determines the connections between them and, through a particular algorithm obtains the immediate glycaemia value.

The device only needs a few seconds to determine the instantaneous glycaemia. This being an absolutely painless and non-invading method, it can be used to get several glycaemia measurements, even in a very short time range, to monitor the trend through time, to check the effectiveness of a therapy, to build glycaemic curves of newborns, children, adults and elderly people.

The values are stored in sequence (up to 5000 assessments) and the memory can be displayed through two slide keys.

The device has a USB gate, a PC connection cable and is provided with a software to visualize and process the data.

Therefore the device is easily activated by pressing the switch-on button. In fact it is enough to put a finger into the device opening and start the centring system to obtain automatically, after a few seconds, the glycaemia value, that will be displayed on the device screen together with date and time of each measurement and the progressive registration number.

Moreover, the selective absorption of the two diode laser rays by the plasmic glucose and the glycate haemoglobin, that works as a control parameter, allows to obtain absolutely accurate glycaemia values.

The two values are calculated in connection with each other, therefore the measurements is absolutely accurate notwithstanding the thickness and the colour of the skin and the thickness of the finger itself.

1. A device to determine the glycaemia value without pricking the skin and without taking a blood sample, comprising:
two laser diode sources for emitting respective laser rays, said two laser sources being operating in a frequency range between 500 and 1000 nm, with a power in the range between 0.01 to 100 mW;
a photodiode sensor for receiving the laser rays emitted by the two laser diodes;
a four-partitioned diaphragm for housing a finger of a patient; and
a display,
wherein the use and working of said device include the following steps:
the patient puts a finger into the device, while operating a lever that opens the four-partitioned diaphragm;
upon releasing the lever, a spring lets the diaphragm close, thus determining the centred arrangement of the finger, do no matter its dimensions, on the focus of the two laser rays emitted by the two laser diodes and directed towards the photodiodes of said sensor;
the patient switches the device on, whereby the passing of the finger sets off an optical barrier that controls the starting of the two laser diodes;
the photodiodes of the sensor read the two values of the laser radiation resulting from the selective absorption of the laser rays, emitted by the two laser diodes, by the glycate haemoglobin and the free plasmic glucose present in the blood of the patient; and
an analogic stage forwards said values to a processor that, after their stabilization, digitises, normalises and extracts a high number of them, strikes an average, and, through a particular algorithm, obtains the instant value of glycaemia, that then appears on the device display,
wherein in the selective absorption of the rays, emitted by said two laser diodes, by the glycate haemoglobin and the free plasmic glucose, the glycate haemoglobin works as a control parameter, so as to obtain absolutely accurate glycaemia values, and
wherein said two values of the laser radiation resulting from the selective absorption of glycate haemoglobin and free plasmic glucose are calculated in connection with each other, whereby the measurement of the glycaemia is absolutely accurate notwithstanding the thickness and the colour of the skin and the thickness of the patient's finger.

2. The device according to claim 1, wherein it only needs a few seconds to determine the instantaneous value of glycaemia;
in an absolutely painless and non-invading method, whereby it can be used to get several glycaemia measurements, even in a very short time range, to monitor the trend through time, to check the effectiveness of a therapy, to build glycaemic curves of newborns, children, adults and elderly people,
wherein the values are stored in sequence up to 5000 assessments and the memory can be displayed through two slide keys, and
wherein the device has a USB gate, a PC connection cable and is provided with a software to visualize and process the data.

3. The device according to claim 2, wherein it is easily activated by pressing the switch-on button, and wherein it is enough to put the patient's finger into the device opening and start the centring system to obtain automatically, after a few seconds, the glycaemia value, that will be displayed on the device screen together with date and time of each measurement and the progressive registration number.
